(19) United States
(12) Patent Application Publication
(10) Pub. No.: US 2021/0275028 A1
(43) Pub. Date: Sep. 9, 2021

(54) IDENTIFYING EXISTENCE OF ORAL DISEASES

(71) Applicant: Vitrix Health INC., Champaign, IL (US)

(72) Inventors: Ayush Kumar, Frisco, TX (US); Aashay Patel, New York, NY (US); Mark Daniel O'Connor, Western Springs, IL (US); William Widjaja, Aurora, IL (US)

(21) Appl. No.: 16/812,269

(22) Filed: Mar. 7, 2020

(51) Int. Cl. A61B 5/00

(52) U.S. Cl. CPC A61B 5/0088; A61B 5/0022; A61B 5/004; A61B 5/0017; A61B 5/7264; A61B 5/4552

(57) ABSTRACT

Aspects of the present disclosure are directed to identifying existence of oral diseases. In an embodiment, a light source operable to provide light in one of multiple of wavelength bands is provided. The light with the corresponding wavelength band of the multiple wavelength bands accentuates a corresponding feature indicative of a respective set of diseases. The light source may be further operated to generate a first light with a desired wavelength band to illuminate a target area in a mouth of a subject, and an image formed by the reflected light from the target area may be examined for the existence of an oral disease corresponding to the desired wavelength band.

### TECHNICAL FIELD

Aspects of the present disclosure relate generally to disease identification, and more specifically to identifying existence of oral diseases.

### RELATED ART

Oral disease refers to a condition manifested in the mouth of an organism (including human beings), and which prevents the body or mind of the organism from working normally. Mouth in turn refers to an anatomical part of the body and generally includes lips, vestibule, mouth cavity, gums, teeth, palate, tongue, salivary glands, etc.

Aspects of the present disclosure relate to identification of existence of oral diseases.

### BRIEF DESCRIPTION OF THE VIEWS OF DRAWINGS

Example aspects of the present disclosure will be described with reference to the accompanying drawings briefly described below.

FIG. 1 is a block diagram of an example device in which several aspects of the present disclosure can be implemented.

FIG. 2 a block diagram illustrating the details of a device for detecting oral disease implemented using an off-the-shelf mobile phone, in an embodiment of the present disclosure.

FIG. 3A depicts the manner in which a control board, lighting system and filter mechanism are placed and connected within a casing, in an embodiment of the present disclosure.

FIG. 3B depicts the details of FIG. 3A with the lighting system and filter mechanism in an assembled state, in an embodiment of the present disclosure.

FIG. 3C depicts a filter mechanism used in a device for detecting oral diseases, in an embodiment of the present disclosure.

FIG. 3D depicts a lighting system with LEDs used in a device for detecting oral diseases, in an embodiment of the present disclosure.

FIG. 3E depicts a camera view port provided in a casing used for housing a device for detecting oral diseases, in an embodiment of the present disclosure.

FIG. 3F depicts an elevation view of a filter mechanism disposed over a camera view port in a device for detecting oral diseases in an embodiment of the present disclosure.

FIG. 3G depicts an elevation view of a filter mechanism and a lighting system disposed over a camera view port in a device for detecting oral diseases in an embodiment of the present disclosure.

FIG. 4 is block diagram illustrating conceptually the operation of a filter mechanism used in a device for detecting oral diseases in an embodiment of the present disclosure.

FIG. 5 is a block diagram depicting the front view of a device used for detecting oral diseases in an embodiment of the present disclosure.

FIG. 6 is a flowchart illustrating the manner in which gum disease (example oral disease) is detected in an embodiment of the present disclosure.

FIG. 7 is a block diagram of further implementation details of the device of FIG. 2 in an embodiment of the present disclosure.

In the drawings, like reference numbers generally indicate identical, functionally similar, and/or structurally similar elements. The drawing in which an element first appears is indicated by the leftmost digit(s) in the corresponding reference number.

### DETAILED DESCRIPTION

### 1. Overview

Aspects of the present disclosure are directed to identifying existence of oral diseases. In an embodiment, a light source operable to provide light in one of multiple of wavelength bands is provided. The light with the corresponding wavelength band of the multiple wavelength bands accentuates a corresponding feature indicative of a respective set of diseases. The light source may be further operated to generate a first light with a desired wavelength band to illuminate a target area in a mouth of a subject, and an image formed by the reflected light from the target area may be examined for the existence of an oral disease corresponding to the desired wavelength band.

According to another aspect of the present disclosure, a mobile phone is provided. The mobile phone includes a light source operable to provide light with one of multiple wavelength bands. The light with the corresponding wavelength band of the multiple wavelength bands accentuates a corresponding feature indicative of a respective disease. The mobile phone further includes a processor to operate the light source to generate a first light with a desired wavelength band to illuminate a target area in a mouth of a subject, and an image capture apparatus to form an image based on the reflected light from the target area.

Several aspects of the present disclosure are described below with reference to examples for illustration. It should be understood that numerous specific details, relationships, and methods are set forth to provide a full understanding of the invention. One skilled in the relevant arts, however, will readily recognize that the invention can be practiced without one or more of the specific details, or with other methods, etc. In other instances, well-known structures or operations are not shown in detail to avoid obscuring the features of the disclosure.

### 2. Example Environment

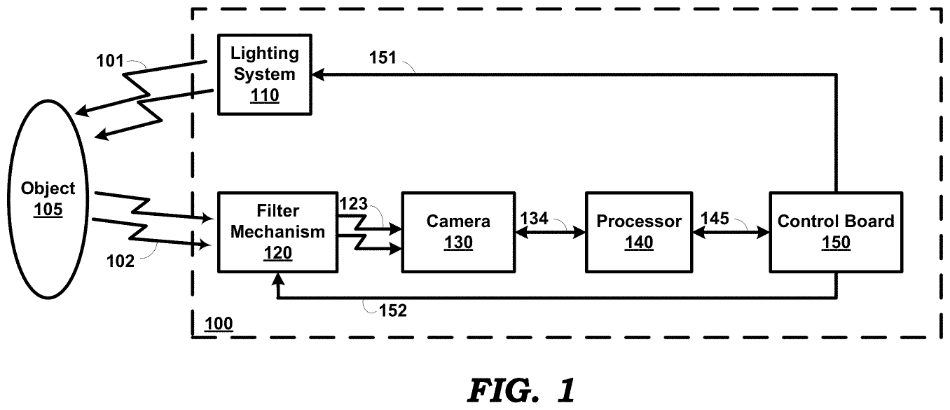

FIG. 1 is a block diagram illustrating an example device in which several aspects of the present disclosure can be implemented. Device 100 is shown containing lighting system 110, filter mechanism 120, camera 130, processor 140 and control board 150. Device 100 may contain other blocks/components (such as those for wired or wireless communication) which are not shown in FIG. 1 in the interest of conciseness. Object 105 represents specific regions in a patient's mouth whose details are to be captured in the form of one or more images by device 100 and analyzed for presence of oral disease.

Lighting system 110 represents one or more light sources. In an embodiment, lighting system 110 consists of concentric rings of light emitting diodes (LED) capable of emitting light of desired wavelength(s) or wavelength band to illuminate object 105. The wavelengths and/or wavelength bands include 405 nanometers (nm), 415 nm, 540 nm, and white light (a band of wavelengths—typically, but not necessarily, of equal intensities—generally in the range between 400 nm and 700 nm). The specific wavelengths or wavelength bands noted above have been chosen for their specific interactions with human tissue. In other embodiments, other light sources/LEDs capable of emitting light of other wavelengths or wavelength bands may be used instead or additionally. The LED(s) can be powered individually, or in combination, via path 151 by control board 150.

Filter mechanism 120 represents a mechanism for selectively positioning a desired one of a set of optical filters in the light path between object 105 and lens of camera 130. Filter mechanism 120 is operable under control from control board 150 (via path 152) to position a filter in front of the lens of camera 130 to filter reflected light 102 and cause filtered light 123 to impinge on optical sensor(s) in camera 130. When no filtering is required, filter mechanism 120 is operable to remove the filter from the light path between object 105 and lens of camera 130, and reflected light 102 directly impinges on the optical sensor(s). In an embodiment, filter mechanism 120 contains a motor which is controllable via path 152 to position the optical filter in, or out of, the light path between object 105 and lens of camera 130. In the embodiment, filter mechanism 120 includes a standard glass filter and a 540 nm band-pass filter. Filter mechanism 120 as suited for specific oral diseases, can be implemented in a known way.

The combination of LED wavelengths and choice of filter may be made to accentuate (be more discernable) one or more features indicative of a corresponding oral disease, and each such combination may be termed as an ‘imaging modality’. For example, and as further described below, control board 110 may use a combination of 405 nm LEDs with a (540±10) nm band-pass filter in the optical path for Auto-fluorescent Imaging (AFI), and use 415 nm and 540 nm LEDs with no filter for Narrow-band Imaging (NBI). Additional filters and wavelength combinations can be used for other imaging modalities. By using multiple modalities in a single device, a multitude of oral diseases can be detected using a single device 100.

Camera 130 includes one or more light sensors (e.g., charge coupled device (CCD), Complementary Symmetry Metal Oxide semiconductor (CMOS) sensors) and other circuitry. The light sensor(s) generate electrical charges in response to light impinging on individual pixels in the sensor(s). The charges are converted into voltages and then a corresponding image (e.g., containing R, G and B components) is generated. When object 105 represents mouth (interior or exterior), camera 130 generates images of the corresponding region in the mouth. Camera 130 forwards each image to processor 140 for further processing.

Processor 140 receives images from camera 130 and operates to determine existence of oral disease from the images, as further described below. Thus, processor 140 may cause the images to be displayed on a display device (not shown in FIG. 1), and indicate existence of oral disease. Although not shown in FIG. 1, device 100 may contain non-volatile memory for storing applications (software applications) that can be executed by processor 140 to determine existence of oral diseases as described herein.

Control board 150 represents electronic circuitry that is capable of controlling lighting system 110 to power a desired set of LEDs and also for controlling filter mechanism 120 to cause a desired filter to be positioned in the light path as noted above. Additionally, control board 150 may provide power for operating lighting system 110 and filter mechanism 120. Control board 130 communicates with processor 140 along bi-directional path 146 to receive commands from processor 140 to control lighting system 110 and filter mechanism 120. Control board 150 can be implemented in a known way.

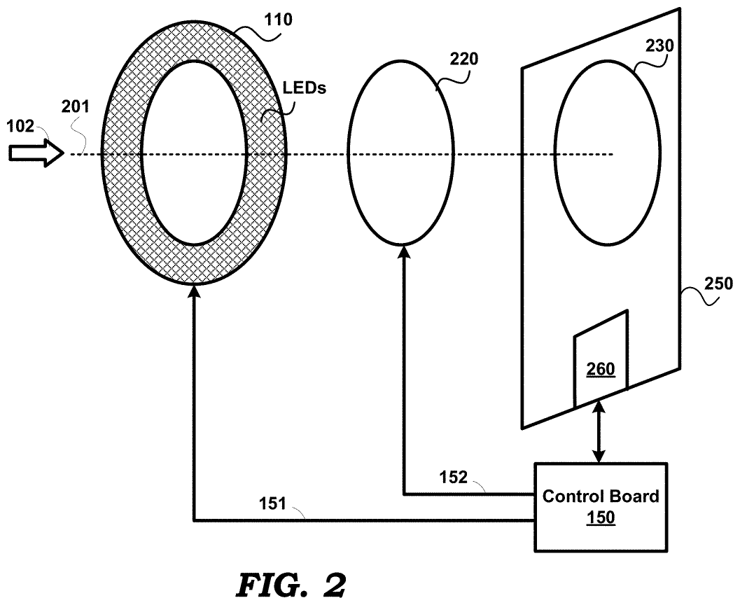

In an embodiment of the present disclosure, device 100 is implemented using an off-the shelf mobile phone, with blocks 110, 120 and 150 suitably coupled to the mobile phone. FIG. 2 is a diagram illustrating the relevant details of such an implementation. FIG. 2 is shown containing mobile phone 150 (with camera port 230 and communications interface 260 shown in FIG. 2), LED PCB assembly 210 and filter 220.

Mobile phone 250 may be an off-the-shelf device, such as, for example a smart phone with an operating system. Applications can be installed and executed in mobile phone 250. Thus, applications for analysis of images captured as noted above, and identification of oral diseases can be developed and installed in mobile phone 250.

Lighting system 110 is shown implemented as a printed circuit board (PCB) containing one or more LEDs arranged as concentric rings on the PCB. Lighting system 110 and filter mechanism 120 (of which only a filter 220 is shown in FIG. 2 in the interest of clarity) are positioned in front of the lens of camera 130. In FIG. 2, blocks 110 and 220 are shown in disassembled condition to indicate that these blocks are concentric and co-axial with respect to camera port 230, and hence with the sensor(s) of camera 130. The common axis 201 also represents the light path of reflected light 102 from object 105 to the sensor(s) of camera 150.

Control board 150 is also shown in FIG. 2. Electronic circuitry in control board 150 is capable of communication with the processor (not shown, but corresponding to processor 140) in mobile phone 250 via communications interface 260, which in the embodiment is a USB (Universal Serial Bus) port. Circuitry in control board 150 also controls the operation of LEDs in lighting system 110 and selection and positioning (or removal) of one or more filters (such as filter 220) in filter mechanism 120.

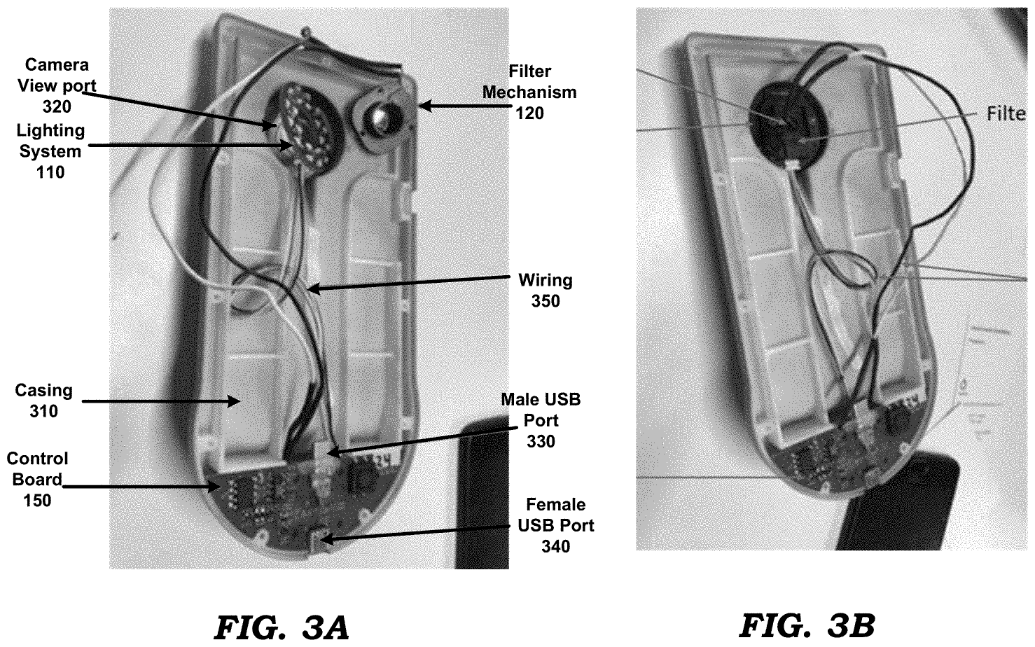

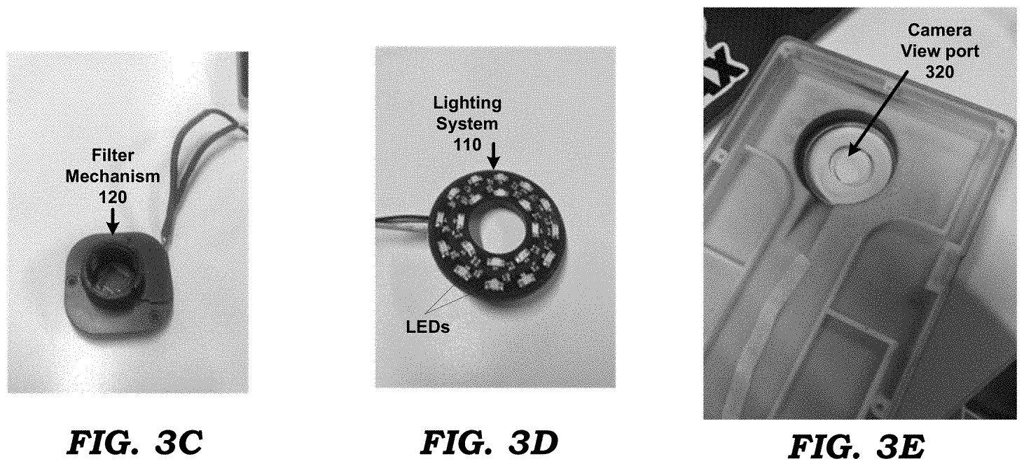

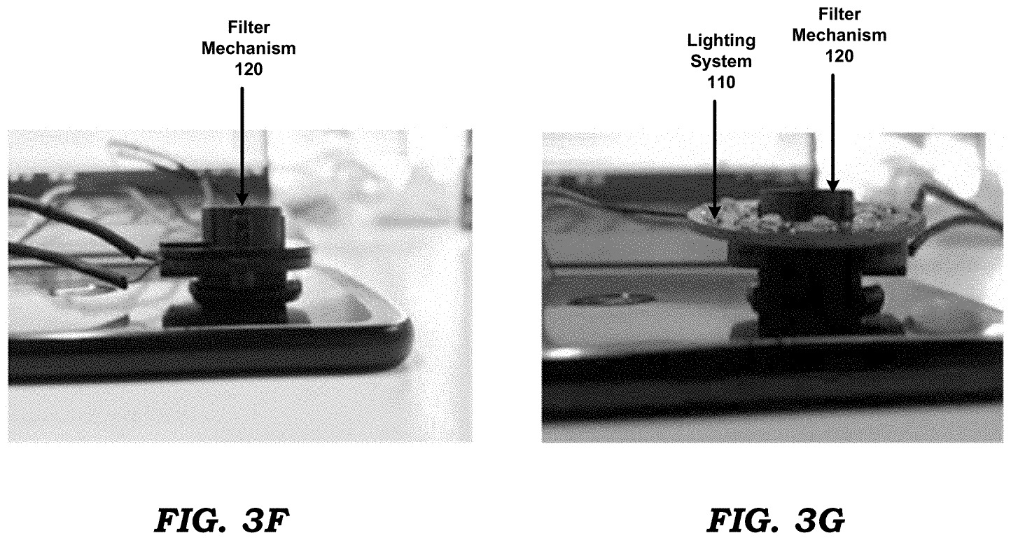

FIGS. 3A-3E depict various views of sections/components/blocks of device 100 in an embodiment. FIG. 3A depicts a casing (310) which houses all the components of device 100, including mobile phone 250, although the phone itself is not shown in FIG. 3A. Camera view port 320, lighting system 110 and filter mechanism 120 are shown in a disassembled state. Control board 150 is shown containing electronic circuitry and male USB port 330 (for communicating with the processor of mobile phone 250) and female USB port 340 for connection to a power supply. Wiring 350 transports power and control signals from control board 150 to lighting system 110 and filter mechanism 120.

FIG. 3B depicts lighting system 110 and filter mechanism 120 assembled and positioned in line with camera view port 320.

FIG. 3C depicts a view of filter mechanism 120. FIG. 3D depicts lighting system 110 in the form of a PCB with LEDs mounted in a concentric ring pattern on the PCB. FIG. 3E depicts camera view port 320.

FIG. 3F depicts filter mechanism 120 positioned over the camera view port. FIG. 3G depicts lighting system 110 fitted around filter mechanism 120.

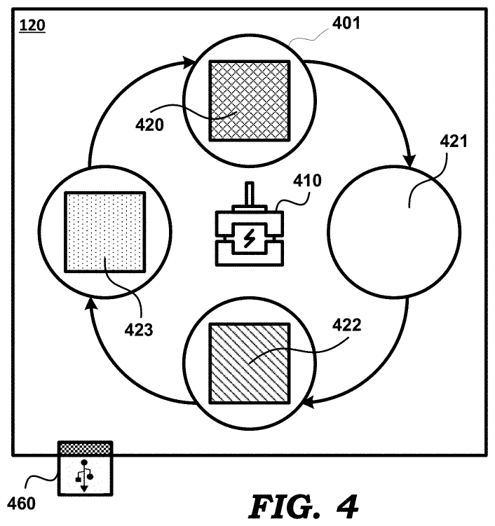

FIG. 4 is a diagram illustrating conceptually the operation of filter mechanism 120. Filter mechanism 120 contains a motor (example stepper motor) 410, filters 420, 421, 422 and 423. Filter 421 is an all-pass filter (effectively implying that no filtering action is performed when this filter is used. Filters 420, 422 and 423 may be low-pass, high-pass or band-pass filters. In an embodiment, one of filters 420, 422 and 423 is a band-pass filtered centered at 540 nm and having a pass band of +/−10 nm. Filter 421 may be implemented as a glass filter. Numeral 401 represents a slot which is aligned with the camera view port and sensor of camera 250. Motor 410 can be commanded by control board 150 to rotate and thereby cause the desired filter to be placed in slot 401. Numeral 460 represents a power connection that is provided via control board 150.

The description is continued with respect to the manner in which device 100 can be operated to acquire images of the mouth area and various imaging modalities that device 100 is capable of.

### 3. Operation

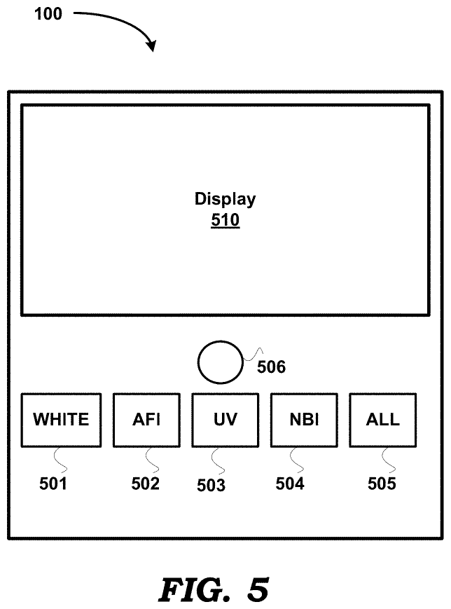

FIG. 5 is an example diagram of the front view of device 100 in an embodiment. Device 100 is shown containing display 510 and user buttons 501-506. Camera view port and camera of device 100 are not shown in FIG. 5 in the interest of conciseness. Application(s) for acquiring image(s) of the mouth regions and processing of the acquired image(s) are assumed to be installed in device 100. Although depicted to be separate from display 510, in an embodiment, user buttons 501-506 are soft buttons displayed on display 510 (which may be touch-sensitive) by the application.

A user operates device 100 to acquire images of the mouth of a patient using camera 130 of device 100. The corresponding one of the installed applications causes a live feed from the camera to be displayed on display 510. User buttons 501, 502, 503, 504 and 505 determine what combination of LED wavelengths for illumination and what filter(s) are to be used while acquiring images, and each user button enables a corresponding “imaging modality”. It should be appreciated that additional modalities can be employed as suited for detection of other oral diseases, with appropriate support of lighting, filters and applications, as will be apparent to a skilled practitioner by reading the disclosure provided herein.

Specifically, pressing user button 501 (WHITE) will power ON (only) white light LED(s) in lighting system 110 and select no filter or all-pass filter 421 in filter mechanism 120. In particular, when the user presses user button 501, the processor in device 100 executing the application senses the pressing, and forwards a corresponding command via communications interface 260 (shown in FIG. 2, and which corresponds to USB interface in an embodiment) to control board 150. In response to the receipt of the command, circuitry in control board 150 causes the white LED(s) to be powered ON and position all-pass filter 421 in the optical path. Accordingly, white light illuminates the mouth region, and the reflected light is captured by camera without filtering to generate an RGB image. This imaging modality is used to detect presence of gum disease in the patient. The generated RGB image is further processed to identify presence of gum disease, as described below.

Pressing user button 502 (AFI) will power ON (only) those LEDs in lighting system 110 that emit light at (or near) 405 nm, and will also position (540 nm+/−10 nm) band pass filter of filter mechanism 130 in the optical path. In particular, when the user presses user button 502, the processor in device 100 executing the application senses the pressing, and forwards a corresponding command via communications interface 260 to control board 150. In response to the receipt of the command, circuitry in control board 150 causes the 405 nm LEDs to be powered ON and positions (540 nm+/−10 nm) band pass filter in the optical path. Accordingly, a narrow band of light at or near 405 nm illuminates the mouth region, and the reflected light is captured by camera after band-pass filtering to generate an image. The image would contain R, G and B components, but the G component will be predominant. This imaging modality is termed auto-fluorescent imaging (AFI), also known as UVC (UV with contrast)), well known in the relevant arts.

Briefly, AFI uses UV (or near-UV) LEDs as the illumination to excite fluorophores within cellular structures as a means of assessing the health of a tissue. More specifically, AFI utilizes near UV LEDs (405 nm, as noted above) to excite fluorophores, dominantly reduced nicotinamide adenine dinucleotide (NADH) and flavin adenine dinucleotide (FAD), which reemit excited photons at a reduced energy level (˜540 nm). This reemitted light then passes through the band-pass filter positioned in front of the camera lens which blocks all light outside the desired wavelength (540±10 nm), showing normal tissue as a bright green. Hyper-keratinized and dysplastic lesions lose fluorescence due to a complex mixture of alterations to intrinsic tissue fluorophore distribution, such as the breakdown of the collagen matrix and elastin composition. This decrease in fluorescence then affects the re-emittance (at ˜540 nm) of the near UV excitation, causing these abnormal tissue sites to appear far darker than their healthy counterparts. Thus, the AVI imaging modality is used for detecting hyper-keratinized and dysplastic lesions in the mouth. Such lesions may indicate tissue where cancer is more likely to occur. They can be the result of burns, cuts, carcinogens, etc.

One type of tissue that can be examined using AVI modality is “stratified squamous epithelium”. It is located in the lining mucosa (comprising of the alveolar mucosa, buccal mucosa, or cheek area), and the masticatory mucosa (hard palate, dorsum of the tongue, and gingiva). The medical term for the lesions noted above is simply “precancerous lesions”, which covers a wide variety of malformations such as leukoplakia, and is a coverall term for an abnormal formation of cells, which may or may not be cancerous.

Pressing user button 503 (UV) will power ON (only) those LEDs in lighting system 110 that emit light at (or near) 405 nm, but does not position any filter in the optical path. This imaging modality may be termed Ultraviolet No Contrast (UV), in which UV LEDs (405 nm) are used to illuminate teeth. In this modality no filter is used. Dentin, a component of the inner tooth, is approximately three times more phosphorescent than the enamel of the tooth. During the formation of dental caries (cavities), decay-causing bacteria make acids that degrade the enamel, thereby exposing the dentin underneath. When a cavity is examined under UV light (405 nm), the dentin fluoresces, thereby enabling easy detection of cavities.

Pressing user button 504 will power ON (only) those LEDs in lighting system 110 that emit light at 415 nm and 540 nm, and will also position all-pass filter of filter mechanism 130 in the optical path. In particular, when the user presses user button 504, the processor in device 100 executing the application senses the pressing, and forwards a corresponding command via communications interface 260 to control board 150. In response to the receipt of the command, circuitry in control board 150 causes the 415 nm and 540 nm LEDs to be powered ON and position all-pass filter (equivalent to no filter at all) in the optical path. Accordingly, a light at 415 nm and 540 nm illuminates the mouth region, and the reflected light is captured by camera without any filtering to generate an image. This imaging modality is termed Narrow-band imaging (NBI), well known in the relevant arts.

Briefly, NBI is an imaging technique for diagnostic or exploratory medical tests, in which light of specific wavelengths (typically blue or green) are used to enhance the detail of certain aspects of the mucosal surface. For the visualization of vascular structures, the necessary wavelengths used are 415 (near UV blue) and 540 (green) nm. These two wavelengths are used as they are the peak absorbances of hemoglobin and penetrate different depths of tissue. 540 nm penetrates to deeper levels of vascular structures while 415 nm highlights capillary groups closer to the mucosal surface. This modality enables identification of the pathological features of mucosal vasculature patterns. In the case of oral mucosal abnormalities, NBI helps identify capillary morphology affected by tumor-induced neovascularization, the rapid production and deformation of the vasculature during tumor formation present in cases of oral cancers, leukoplakia, erythroplakia, and others.

Pressing user button 505 (ALL), will cause device 100 to capture an image in each modality in a serial fashion. Thus, device 100 captures an image in “white’ imaging modality, AFI, UV, and finally NBI modality. Pressing user button 506 causes device 100 to capture more images in only the ‘current’ imaging modality (i.e., the currently selected modality).

It may be appreciated that each imaging modality accentuates one or more features indicative of corresponding disease. For example, images captured using NBI modality accentuates oral mucosal abnormalities. Images captured using AFI accentuate Hyper-keratinized and dysplastic lesions, etc.

The images obtained using the imaging modalities as described herein may be further processed by the corresponding application executed on the mobile phone to determine presence or absence of corresponding oral diseases. Alternatively, or additionally, the images may be transmitted by the mobile phone to servers on the cloud for such determination.

For detecting gum disease, as noted above, the user presses user button 501. The captured RGB image is further processed to detect gum disease as described next with respect to a flowchart.

### 4. Detecting Gum Disease

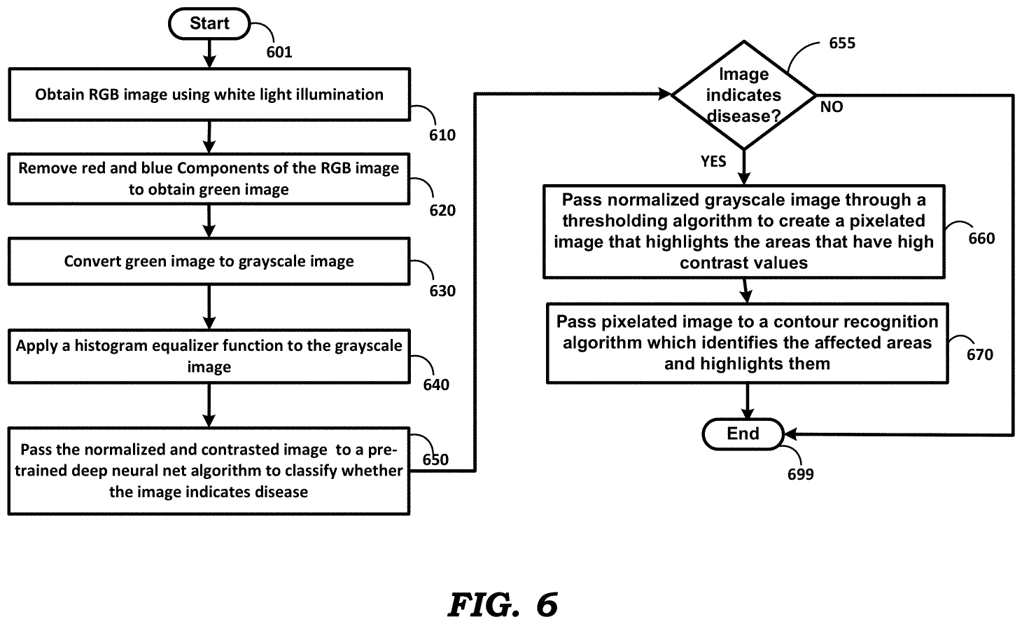

FIG. 6 is a flowchart illustrating the manner in which gum disease (example oral disease) is detected, according to an aspect of the present disclosure. The flowchart is described with respect to device 100 (specifically the application executing in device 100) merely for illustration. However, many of the features can be implemented in other environments (and using potentially other types of systems/devices) also without departing from the scope and spirit of several aspects of the present disclosure, as will be apparent to one skilled in the relevant arts by reading the disclosure provided herein.

In addition, some of the steps may be performed in a different sequence than that depicted below, as suited to the specific environment, as will be apparent to one skilled in the relevant arts by reading the present disclosure. Many of such implementations are contemplated to be covered by several aspects of the present disclosure. The flow chart begins in step 601, in which control immediately passes to step 610.

In step 610, device 100 obtains an RGB image of a mouth region using white light illumination, as described above. Control then passes to step 620.

In step 620, device 100 removes red and blue components of the RGB image to obtain a green image. Control then passes to step 630.

In step 630, device 100 converts the green image to a grayscale image. Control then passes to step 640.

In step 640, device 100 applies a histogram equalizer function to the grayscale image. The application of the histogram equalizer function causes equalization and normalization of the grayscale image, and increase the contrast in the grayscale image. Typically, the pixel values obtained from step 630 are confined to some specific range of values only. Hence, brighter images will have all pixels confined to high values. The histogram equalizer function improves the contrast values by “spreading out” this pixel values over a larger value range. The histogram equalization may be implemented, for example, using “Open Source Computer Vision Library”, available from public sources. Control then passes to step 650.

In step 650, the normalized and contrasted image obtained from step 640 is passed to a pre-trained deep neural net algorithm to classify whether the image indicates presence of disease. The deep neural net algorithm uses a supervised CNN (convolutional neural network) that has been trained using images from healthy and unhealthy patients. The algorithm is first trained by collecting a dataset of oral images and categorizing them as healthy or diseased. These images are then fed into the CNN algorithm which then uses a series of neurons and layers of neurons to come to a consensus on how to categorize these images. This trained algorithm is then run in the future to categorize future images the algorithm sees. The CNN algorithm may be implemented, for example, as described in “Convolutional Neural Network” available with Stanford University, 450 Jane Stanford Way, Stanford, Calif. 94305-2004. Control then passes to step 655.

In step 655, device 100 determines if the operation of step 650 classified the image as indicative of disease. If disease is indicated, control passes to step 660, and otherwise to step 699.

In step 660, the normalized grayscale image (obtained in step 650) is passed through a thresholding algorithm to create a pixelated image that highlights the areas that have high contrast values. The thresholding algorithm may be implemented, for example, using Open Source Computer Vision Library”, available from public sources. Control then passes to step 670.

In step 670, the pixelated image obtained from step 660 is passed to a contour recognition algorithm which identifies the diseased areas in the image and highlights them. A clinician can interpret the image and advise the patient as to the next steps. Control then passes to step 699, in which the flowchart ends.

It is noted here that all the steps of flowchart of FIG. 6 may be performed by the processor in the mobile phone. Alternatively, the processor may perform only the operation of step 610, and then cause the image to be transmitted to a cloud server, which would then perform the other steps.

According to another aspect of the present disclosure, an application is designed to identify a patient's risk of having oral disease as described next.

During each oral screening, an application (termed Vitrix Health Risk Stratification algorithm) executed on the mobile phone processes the historical data of a patient. During each visit, the patient is asked the below questions to ensure that the data has not changed. The questions with the possible responses are noted below:

1. Smoking
Yes or No

2. Chewing tobacco
Yes or No

3. Chewing quid with tobacco
Yes or No

4. Chewing quid without tobacco
Yes or No

5. Alcohol
Yes or No

6. Fruit consumption
Yes or No

7. Family history of cancer
Yes or No

8. Rinsing mouth after eating
Yes or No

The patient's responses to the above-noted questions are fed it into the following risk equation:

ocr=(w1*s)+(w2*ct)+(w3*cqwt)+(w4*cqnt)+(w5*al)+(w6*fc)+(w7*fh)+(w8*rm)

wherein,

s=Smoking,

ct=Chewing tobacco,

cqwt=Chewing quid with tobacco,

cqnt=Chewing quid without tobacco,

al=Alcohol,

fc=Fruit consumption,

fh=Family history of cancer,

rm=Rinsing mouth after eating,

ocr=Oral Cancer Risk, and

w1 through w8 are corresponding weights.

The above equation was created based on data from other oral cancer patients. In the equation above, the weights w1-w8 are pre-calculated using a regression technique. After adding the responses to the questions into the equation, the magnitude of ‘ocr’ is computed by the processor in the mobile phone, and the result is interpreted by the clinician to see how much risk the patient has of getting oral cancer and then proceeding accordingly (either performing a screening or not screening the patient).

An electronic health record (EHR) can be created from the images and data obtained for a patient. The Electronic Health Record system, which might be located in a cloud, allows the clinician to store the images taken during an oral screening, manage patients, and generate PDF reports that can be sent to dentists or given to the patient. The process of how this works is listed below:

1. The clinician takes photos of the patient's mouth using the device 100.

2. The clinician then tags the image with the type of image (narrowband, UV light, or white light).

3. The clinician adds comments to the photos.

4. The photos, with the attached comments, are sent to a server that stores them in the clinician profile.

5. The clinician can access their profile on a web portal and see all their patients.

6. On the web portal, the clinician can see all the past screening images and the comments associated with them.

7. The clinician can then create a PDF document that can be downloaded and sent to dentists or the patient.

The description is continued with an illustration of the implementation details of device 100 (including the portions of the off-the-shelf smart phone 250 of FIG. 2) in an embodiment of the present disclosure

### 5. Device/Mobile Phone

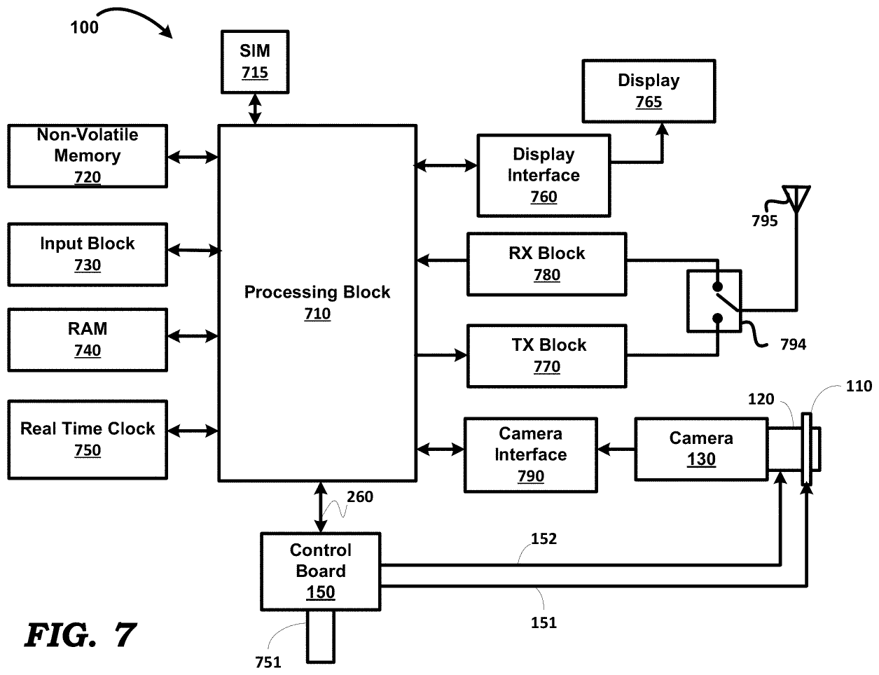

FIG. 7 is a block diagram of a device in an embodiment of the present disclosure. In addition to blocks 110, 120, 130 and 150 of FIG. 1, FIG. 7 depicts relevant parts/blocks of an off-the-shelf smart phone that are contained in the device. Device 100 (which may itself be referred to as a mobile phone) of FIG. 7 is shown containing processing block 710, SIM 715, non-volatile memory 720, input/output (I/O) block 730, random access memory (RAM) 740, real-time clock (RTC) 750, display interface 760, display 765, transmit (Tx) block 770, receive (Rx) block 780, switch 790, and antenna 495. Some or all blocks of device 100 may be powered by a battery (not shown). In another aspect of the present disclosure, device 100 is mains-powered and contains corresponding components such regulators, filters, etc. (not shown). The specific blocks of device 100 are shown by way of illustration only, and device 100 may contain more or fewer blocks depending on specific requirements.

SIM 715 represents a subscriber identity module (SIM) that may be provided by a network operator. A SIM may store the international mobile subscriber identity (IMSI) number (also the phone number) used by a network operator to identify and authenticate a subscriber. Additionally, a SIM may store address book/telephone numbers of subscribers, security keys, temporary information related to the local network, a list of the services provided by the network operator, etc. Though not shown, device 100 may be equipped with a SIM card holder for housing SIM 715. Typically, the SIM is ‘inserted’ into such housing before the device can access the services provided by the network operator for subscriber configured on the SIM. Processing block 710 may read the IMSI number, security keys etc., in transmitting and receiving voice/data via Tx block 470 and RX block 480 respectively. SIM 715 may subscribe to data and voice services according to one of several radio access technologies such as GSM (Global System for Mobile Communications), LTE (Long Term Evolution, FDD as well as TDD), CDMA (Code Division Multiple Access), WCDMA (Wideband CDMA), 5G, etc.

RTC 750 operates as a clock, and provides the ‘current’ time to processing block 710. Additionally, RTC 750 may internally contain one or more timers. Input block 730 provides interfaces for user interaction with device 100, and includes input devices. The input devices may include a keypad and a pointing device (e.g., touch-pad).

Antenna 795 operates to receive from, and transmit to, a wireless medium, corresponding wireless signals (representing voice, data, etc.) according to one or more standards such as LTE (Long Term Evolution). Switch 794 may be controlled by processing block 710 (connection not shown) to connect antenna 795 to one of blocks 770 and 780 as desired, depending on whether transmission or reception of wireless signals is required. Switch 794, antenna 795 and the corresponding connections of FIG. 7 are shown merely by way of illustration. Instead of a single antenna 795, separate antennas, one for transmission and another for reception of wireless signals, can also be used.

TX block 770 receives, from processing block 710, digital signals representing information (voice, data, etc.) to be transmitted on a wireless medium (e.g., according to the corresponding standards/specifications), generates a modulated radio frequency (RF) signal (according to the standard), and transmits the RF signal via switch 794 and antenna 795. TX block 770 may contain RF circuitry (mixers/up-converters, local oscillators, filters, power amplifier, etc.) as well as baseband circuitry for modulating a carrier with the baseband information signal. Alternatively, TX block 770 may contain only the RF circuitry, with processing block 710 performing the modulation and other baseband operations (in conjunction with the RF circuitry). Images, text, data (e.g., electronic health record described above), etc., generated as described above can be transmitted by TX block 770 to cloud servers under control from processing block 710.

RX block 780 represents a receiver that receives a wireless (RF) signal bearing voice/data and/or control information via switch 794, and antenna 795, demodulates the RF signal, and provides the extracted voice/data or control information to processing block 710. RX block 780 may contain RF circuitry (front-end filter, low-noise amplifier, mixer/down-converter, filters) as well as baseband processing circuitry for demodulating the down-converted signal. Alternatively, RX block 780 may contain only the RF circuitry, with processing block 710 performing the baseband operations in conjunction with the RF circuitry

Non-volatile memory 720 is a non-transitory machine readable medium, and stores instructions (forming one or more of the applications noted above as well as operating systems, such as Android OS), which when executed by processing block 710, causes device 100 to operate as described herein. In particular, the instructions enable device 100 to capture images according to the various imaging modalities described above, as well as perform various processing operations described above, including those of flowchart 6. The instructions may either be executed directly from non-volatile memory 720 or be copied to RAM 740 for execution.

RAM 740 is a volatile random access memory, and may be used for storing instructions and data. RAM 740 and non-volatile memory 720 (which may be implemented in the form of read-only memory/ROM/Flash etc.) constitute computer program products or machine (or computer) readable medium, which are means for providing instructions to processing block 710. Processing block 710 may retrieve the instructions, and execute the instructions to provide several features of the present disclosure.

Display interface 760 contains circuitry to enable processing block 710 to drive display 765 to cause images, text, etc. to be displayed on display 765. Display 765, which corresponds to display 510 of FIG. 5, displays data in the form of images or text using technologies such as for example LCD (Liquid Crystal Display), LED (Light Emitting Diode), OLED (Organic LED), etc., and may also contain a touch sensitive surface implemented using corresponding technologies, such as capacitive touch sensing.

Camera interface 790 contains circuitry to enable processing block 710 to interface with camera 130 (also shown in FIG. 1), and thereby capture images of objects. Lighting system 110 and filter mechanism 120 (of FIG. 1) are shown fitted to camera 130.

Control board 150 (of FIGS. 1 and 2) is shown connected to lighting system 110 and filter mechanism 120 via respective paths 151 and 152, and controls these units as described in details above. Control board 150 is shown coupled to processing block 710 via communications interface 260 (also shown in FIG. 2), which may correspond to a USB port in an embodiment. Control board 150 contains communications port 751 to enable transfer of power from an external power source (not shown) and transfer of data to/from external devices. Power from an external power source may be used to charge batteries (not shown) in device 100, and also power LEDs in lighting system 110 and to operate filter mechanism 120.

Processing block 710 (or processor in general) corresponds to processor 140 of FIG. 1, and may contain multiple processing units internally, with each processing unit potentially being designed for a specific task. Thus, processing block 710 may be implemented as multiple separate processing cores, each for executing corresponding execution threads of corresponding software application(s). Alternatively, processing block 710 may represent a single processing unit executing multiple execution threads representing one or more software applications. In general, processing block 710 executes instructions stored in non-volatile memory 750 or RAM 740 to enable device 100 to operate according to several aspects of the present disclosure, described in detail herein. Alternatively, processing block 710 may only cause the images of mouth regions of a person to be captured, and transmit the images (including additional data as appropriate) to a cloud server, where processors there may examine the images to determine presence or absence of oral diseases as indicated by the images.

### 6. Conclusion

References throughout this specification to “one aspect of the present disclosure”, “an aspect of the present disclosure”, or similar language means that a particular feature, structure, or characteristic described in connection with the aspect of the present disclosure is included in at least one aspect of the present disclosure of the present invention. Thus, appearances of the phrases “in one aspect of the present disclosure”, “in an aspect of the present disclosure” and similar language throughout this specification may, but do not necessarily, all refer to the same aspect of the present disclosure.

While various aspects of the present disclosure have been described above, it should be understood that they have been presented by way of example only, and not limitation. Thus, the breadth and scope of the present disclosure should not be limited by any of the above-described aspects, but should be defined only in accordance with the following claims and their equivalents.

What is claimed is:

1. A method of identifying existence of oral diseases, the method comprising:
providing a light source operable to provide light in one of a plurality of wavelength bands, wherein the light with the corresponding wavelength band of the plurality of wavelength bands accentuates a corresponding feature indicative of a respective set of diseases;
operating said light source to generate a first light with a desired wavelength band to illuminate a target area in a mouth of a subject; and
examining an image formed by the reflected light from the target area for the existence of an oral disease corresponding to the desired wavelength band.

2. The method of claim 1, wherein said providing and said operating are performed in a mobile phone.

3. The method of claim 2, wherein said examining is also performed in said mobile phone.

4. The method of claim 3, further comprising:
operating a filter switching mechanism to position an optical filter in the path of said reflected light,
wherein the filter additionally accentuates said corresponding feature.

5. The method of claim 4, wherein said respective disease is one of tooth cavity, gum disease, oral mucosal abnormality, and hyper-keratinized and dysplastic lesions.

6. The method of claim 5, wherein said operating operates said light source to generate said first light with a wavelength of about 405 nanometers (nm) and said operating a filter switching mechanism positions a band-pass optical filter having a pass band centered at around 540 nm in the path of said reflected light to enable identification of said hyper-keratinized and dysplastic lesions,
wherein said operating operates said light source to generate said first light with wavelengths of 415 nm and 540 nm, and said operating a filter switching mechanism positions an all-pass optical filter in the optical path of said reflected light to enable identification of said oral mucosal abnormality.

7. The method of claim 5, wherein said operating operates said light source to generate white light, and said operating a filter switching mechanism positions an all-pass optical filter in the light path of said reflected light to generate a red, green, blue (RGB) image to enable identification of said gum disease.

8. The method of claim 7, wherein said identification of said gum disease further comprises:
removing red and blue components of said RGB image to obtain a green image;
converting said green image to a grayscale image;
applying a histogram equalizer function to said grayscale image to cause equalization and normalization of said grayscale image, and increase contrast in the grayscale image, said applying said histogram equalizer function generating a normalized and contrasted image;
passing said normalized and contrasted image to a pre-trained deep neural net algorithm to classify whether said normalized and contrasted image indicates presence of disease, wherein said deep neural net algorithm uses a supervised CNN (convolutional neural network);
if said pre-trained deep neural net algorithm indicates presence of disease, then passing said normalized and contrasted image through a thresholding algorithm to create a pixelated image that highlights the areas that have high contrast values; and
passing said pixelated image to a contour recognition algorithms to identify and highlight diseased areas in said pixelated image.

9. The method of claim 8, further comprising computing a patient's risk of oral cancer according to an equation: ocr=(w1*s)+(w2*ct)+(w3*cqwt)+(w4*cqnt)+(w5*al)+(w6*fc)+(w7*fh)+(w8*rm),
wherein s equals 1 if said patient smokes and equals 0 if said patient does not smoke,
wherein ct equals 1 if said patient chews tobacco and equals 0 if said patient does not chew tobacco,
wherein cqwt equals 1 if said patient chews quid with tobacco and equals 0 if said patient does not chew quid with tobacco,
wherein cqnt equals 1 if said patient chews quid without tobacco and equals 0 if said patient does not chew quid without tobacco,
wherein al equals 1 if said patient consumes alcohol and equals 0 if said patient does not consume alcohol,
wherein fc equals 1 if said patient consumes fruit and equals 0 if said patient does not consume fruit,
wherein fh equals 1 is said patient has a family history of cancer and equals 0 if said patient does not have a family history of cancer,
wherein rm equals 1 if said patient does not rinse mouth after eating and equals 0 if said patient rinses mouth after eating,
wherein ocr equals patient's risk of oral cancer,
wherein said equation is based on data from other oral cancer patients, and
w1, w2, w3, w4, w5, w6, w7 and w8 are weights pre-calculated using a regression technique.

10. A mobile phone comprising:
a light source operable to provide light with one of a plurality of wavelength bands, wherein the light with the corresponding wavelength band of the plurality of wavelength bands accentuates a corresponding feature indicative of a respective disease;
a processor to operate said light source to generate a first light with a desired wavelength band to illuminate a target area in a mouth of a subject; and
an image capture apparatus to form an image based on the reflected light from the target area.

11. The mobile phone of claim 10, wherein the processor is operable to examine an image formed by the reflected light from the target area for the existence of an oral disease corresponding to the desired wavelength band.

12. The mobile phone of claim 10, wherein said processor executes a software application which provides a user interface for a user to specify said desired wavelength band, said mobile phone further comprising:
a control board to receive a command from said processor and to control said light source to generate said first light with said desired wavelength band,
wherein said processor receives a user input specifying said desired bandwidth from said user interface and generates said command.

13. The mobile phone of claim 12, further comprising a filter switching mechanism comprising a plurality of optical filters, wherein said processor is further operable to cause said filter switching mechanism to position a first filter of said plurality of optical filters in the path of said reflected light,
wherein said first filter additionally accentuates said corresponding feature,
wherein said user input also specifies said first filter,
wherein said control board is further designed to control said filter switching mechanism to position said first filter in the path of said reflected light in response to receipt of said command from said processor.

14. The mobile phone of claim 13, wherein said respective disease is one of tooth cavity, gum disease, oral mucosal abnormality, and hyper-keratinized and dysplastic lesions.

15. The mobile phone of claim 14, wherein said processor is designed to operate said light source to generate said first light with a wavelength of 405 nanometers (nm) and position a band-pass optical filter having a pass band centered at 540 nm in the path of said reflected light to enable identification of said hyper-keratinized and dysplastic lesions.

16. The mobile phone of claim 14, wherein said processor is designed to operate said light source to generate said first light with wavelengths of 415 nm and 540 nm, and position an all-pass optical filter in the optical path of said reflected light to enable identification of said oral mucosal abnormality.

17. The mobile phone of claim 14, wherein said processor operates said light source to generate white light, and position an all-pass optical filter in the light path of said reflected light to generate a red, green, blue (RGB) image to enable identification of said gum disease.

18. The mobile phone of claim 17, wherein to enable identification of said gum disease, said processor is further operable to:
remove red and blue components of said RGB image to obtain a green image;
convert said green image to a grayscale image;
apply a histogram equalizer function to said grayscale image to cause equalization and normalization of said grayscale image, and increase contrast in said grayscale image to generate a normalized and contrasted image;
pass said normalized and contrasted image to a pre-trained deep neural net algorithm to classify whether said normalized and contrasted image indicates presence of disease, wherein said deep neural net algorithm uses a supervised CNN (convolutional neural network);
if said pre-trained deep neural net algorithm indicates presence of disease, then to pass said normalized and contrasted image through a thresholding algorithm to create a pixelated image that highlights the areas that have high contrast values; and
pass said pixelated image to a contour recognition algorithms to identify and highlight diseased areas in said pixelated image.

19. A non-transitory machine readable medium storing one or more sequences of instructions for enabling a user to identify existence of oral diseases using a system, wherein execution of said one or more instructions by one or more processors contained in said system enables said system to perform the actions of:
providing a light source operable to provide light in one of a plurality of wavelength bands, wherein the light with the corresponding wavelength band of the plurality of wavelength bands accentuates a corresponding feature indicative of a respective set of diseases;
operating said light source to generate a first light with a desired wavelength band to illuminate a target area in a mouth of a subject; and
examining an image formed by the reflected light from the target area for the existence of an oral disease corresponding to the desired wavelength band.

20. The non-transitory machine readable medium of claim 19, wherein said system is a mobile phone, said non-transitory machine readable medium further comprising instructions for:
operating a filter switching mechanism to position an optical filter in the path of said reflected light,
wherein the filter additionally accentuates said corresponding feature.
